(19) United States
(12) Patent Application Publication
(10) Pub. No.: US 2026/0364004 A1
(43) Pub. Date: Nov. 19, 2026

(54) AN ORALLY DISINTEGRATING TABLET CONTAINING GABAPENTIN OR PHARMACEUTICALLY ACCEPTABLE SALTS THEREOF AND THE PROCESS OF PREPARING THE SAME

(71) Applicant: NOVUMGEN LIMITED, London (GB)

(72) Inventor: Kamleshkumar Patel, London (GB)

(21) Appl. No.: 19/116,282

(22) PCT Filed: Dec. 21, 2023
(86) PCT No.: PCT/GB2023/053353

§ 371 Date: Mar. 27, 2025

(30) Foreign Application Priority Data
Dec. 21, 2022 (GB) 2219356.9

(51) Int. Cl. A61K 9/00; A61K 9/20; A61K 31/195

(52) U.S. Cl. CPC A61K 9/0056; A61K 9/2013; A61K 9/2027; A61K 9/2031; A61K 9/2054; A61K 9/2059; A61K 9/2095; A61K 31/195

(57) ABSTRACT

The present invention relates to an orally disintegrating tablet comprising Gabapentin or pharmaceutically acceptable salts thereof, at least one diluent, a disintegrant, a stabilizer, a binder, a sweetener, and a lubricant. The orally disintegrating tablet provides faster disintegration of gabapentin upon contact with saliva in the buccal cavity. The orally disintegrating tablet of gabapentin is effective improving Patient Compliance, and accurate dosing as compared to liquids, to overcome swallowing challenges for elderly, bed-ridden, and psychiatric patients.

### FIELD OF THE INVENTION

The present invention relates to a pharmaceutical composition of Gabapentin. The present invention relates to an orally disintegrating tablet of Gabapentin or pharmaceutically acceptable salts thereof for oral administration. The present invention also relates to the process of the preparation of the same.

### BACKGROUND OF THE INVENTION

Gabapentin was first disclosed in the U.S. Pat. No. 4,024,175. Gabapentin is a structural analogue of the inhibitory neurotransmitter gamma-aminobutyric acid (GABA).

The primary mode of action appears to be at the auxiliary α2δ-1 subunit of voltagegated calcium channels. The major function of these subunits is to facilitate the movement of pore-forming α2δ-1 subunits of calcium channels from the endoplasmic reticulum to the cell membrane of pre-synaptic neurons. Gabapentin appears to inhibit the action of α2δ-1 subunits, thus decreasing the density of pre-synaptic voltage-gated calcium channels and the subsequent release of excitatory neurotransmitters.

Gabapentin is indicated for the treatment of postherpetic neuralgia and various types of peripheral neuropathic pain, such as painful diabetic neuropathy in adults 25 and partial-onset seizures in paediatrics.

The IUPAC name of gabapentin is 2-[1-(amino methyl)cyclohexyl]acetic acid. The plasma half-life of gabapentin in patients with normal renal function is 5-7 hours. Gabapentin is a high-water solubility and low permeability drug which makes it belong to BCS Class-Ill in the Biopharmaceutical classification system.

Gabapentin is marketed, particularly as an oral tablet, capsule, or solution. The commercially marketed products of Gabapentin in tablet form are available in five dosage strengths: 100, 300, 400, 600, and 800 mg for oral administration. Orally disintegrating tablets provides a good mouth feel property that improves patient compliance, particularly for paediatric patients. Moreover, Gabapentin is a freely water-soluble drug, making it a suitable drug for orally disintegrating tablet.

The marketed tablet of gabapentin is available in large size which is difficult to swallow for elderly, paediatrics resulting in patient noncompliance. Therefore, Gabapentin is a good candidate for formulating the orally dispersible tablet as it is easy to swallow and would provide patient compliance for paediatrics, geriatric and psychiatric patients disabled bedridden patients, and for traveling and busy people, who do not have ready access to water or difficulty in swallowing.

The orally disintegrating tablet of the present invention is a pharmaceutical formulation that rapidly disintegrates in the mouth which helps in easy administration of tablet and provide accurate dosing as compared to liquid formulations.

EP1784174A1 discloses a stable pharmaceutical composition comprising gabapentin as the active ingredient, tribasic calcium phosphate as a sliding agent, hydrogenated castor oil as a lubricant, sorbitol and alginic acid in mixture as diluents. The manufacturing process of invention involves a mixture of powders of gabapentin and inactive ingredients are mixed properly in a mixer for 10 minutes at room temperature. The final powder mixture thus obtained are filled into capsules.

EP2923694A1 discloses a liquid pharmaceutical solution comprising gabapentin or a pharmaceutically acceptable salt, solvate, or hydrate thereof and one or more pharmaceutically acceptable excipients. The manufacturing process of this invention is carried out under nitrogen gas as follows: A Tank filled with water, to this Maltitol solution, is added. Thereafter, sodium benzoate and disodium EDTA as an antioxidant are added to this solution and mixed to obtain a homogeneous mixture and then Gabapentin is added to this pre-mixed solution. Mixing other excipients to the final solution to achieve complete dissolution of ingredients. The final mixture is filtrated and filled into bottles.

EP1558218A1 discloses the conventional tablet comprising gabapentin and one or more excipients. The manufacturing process of this invention involves a wet granulation method as per following steps: a first portion of a binder is added to dried mixture of gabapentin and one or more excipients as combination or in separate way. To the dried granules, a second portion of binder in solution or dispersion form is added, followed by drying granules and then compressing dried granules into tablets.

EP1513504A1 discloses the sustained-release tablet comprises gabapentin or a pharmaceutically acceptable salt or hydrates thereof and at least one rate-controlling polymer. The manufacturing process of this invention involves forming granules by mixing gabapentin with rate-controlling polymer and inactive ingredients, a water or binder solution is added to pre-mixed granules followed by drying the wet granules and compressed into tablets which provides gabapentin release for up to 12 hours.

US20090176882A1 discloses the gastric retentive oral dosage form of gabapentin for once or twice daily dose administration for improving patient compliance. The process of this invention involves dispersing gabapentin in hydrophilic polymer that upon ingestion into gastric fluid swells to a size sufficient to achieve retention of the dosage form in the stomach in a fed mode for a period of at least about five hours

### SUMMARY OF THE INVENTION

The present invention provides an orally disintegrating tablet that comprises Gabapentin or pharmaceutically acceptable salts thereof, Gabapentin is a good candidate for formulating orally disintegrating tablet that can provide good palatability and taste-masking properties for improving patient compliance.

Another embodiment of the invention provides the orally disintegrating tablet containing Gabapentin or pharmaceutically acceptable salts thereof, the diluent, and at least one or more ingredient such as a binder, a disintegrant, a stabilizer, a lubricant and a sweetener.

Further, another embodiment of the present invention that involves a process for preparing the orally disintegrating tablet. The orally disintegrating tablet is manufactured by the direct compression method.

Another embodiment of the present invention can effectively treat postherpetic neuralgia and various types of peripheral neuropathic pain, such as painful diabetic neuropathy in adults and partial-onset seizures in paediatrics.

### OBJECTS OF THE INVENTION

The principal object of the present invention is to provide an orally disintegrating tablet of Gabapentin or pharmaceutically acceptable salts thereof.

It is further another object of the present invention to provide fast disintegration and dissolution of the dosage form as it gets in contact with saliva.

Yet another object of the present invention is to provide taste-masking properties and present pleasant palatability for paediatrics and also for administration to patients who cannot swallow, like the elderly, stroke victims, and bedridden patients; who do not have ready access to water.

### DETAILED DESCRIPTION

The present invention is an orally disintegrating tablet suitable for oral administration comprising gabapentin or pharmaceutically acceptable salts thereof with fast disintegrating properties as compared to conventional solid dose forms.

As per one embodiment of the present invention, gabapentin or pharmaceutically acceptable salts thereof, is present in the range of about 30% w/w to about 70% w/w, preferably in the range of about 40% w/w to about 60% w/w.

The term “about”, as and when used in this specification, means±10% of the mentioned value.

As per one embodiment of the present invention, the composition comprises a diluent selected from the group consisting of spray-dried lactose, dicalcium phosphate, dextrates, microcrystalline cellulose, dextrose, maize starch, fructose, Sorbitol, pregelatinized starch, starch, xylitol, sucrose, Sorbitol, maltodextrin, maltose, mannitol or combinations thereof. In the present invention, combination of microcrystalline cellulose and maize starch is preferred as a diluent in the range of about 15% w/w to about 55% w/w, preferably in the range from about 30% w/w to about 45% w/w. Microcrystalline cellulose is present in the range of about 15% w/w to about 50% w/w, preferably in the range from about 20% w/w to about 40% w/w and maize starch is present in the range from about 5% w/w to about 30% w/w, preferably in the range from about 7.0% w/w to about 25.0% w/w. Maize Starch is used as a diluent as starches are known to swell upon contact with water through a wicking mechanism. Microcrystalline cellulose has a fast-wicking rate of water and small elastic deformation. The combination of microcrystalline cellulose and maize starch as diluents can provide fast disintegrating properties to the tablets.

As per another embodiment of the present invention, 30% of total weight of microcrystalline cellulose, which is characterized in that the average particle size of the microcrystalline cellulose particle is the range from about 70 μm to about 100 μm, preferably from about 90 μm to about 100 μm and the bulk density is in the range from about 0.26 g/ml to about 0.34 g/ml, preferably in the range from about 0.30 g/ml to about 0.33 g/ml and remaining 70% of microcrystalline cellulose, which is characterized in that the particle size of the microcrystalline cellulose is lesser than 75 μm preferably lesser than 70 μm and the bulk density is in the range from about 0.10 g/cm³ to about 0.15 g/cm³, preferably in the range from about 0.11 g/cm³ to about 0.14 g/cm³.

As per one more embodiment of the present invention, the disintegrant selected from the group consisting of Modified starches, cross-linked polyvinylpyrrolidone, croscarmellose sodium, soy polysaccharide, Cross-linked alginic acid, gellan gum, calcium Silicate or combinations thereof. Crospovidone is preferred as a disintegrant for the present invention in the range from about 0.5% w/w to about 10.0% w/w, preferably in the range from about 1.5% w/w to about 7.5% w/w. Crospovidone quickly wicks saliva into the tablet to generate the volume expansion and hydrostatic pressures necessary to provide rapid disintegration in the mouth

As per one embodiment of the present invention, a stabilizing agent selected from the group consisting of Poloxamers, Amino acid-based stabilizers, sodium alginate, sodium carboxymethyl cellulose (CMC), guar gum, locust bean gum, carrageenan, gelatin, and pectin, Povidone, Polyvinyl alcohol, sodium lauryl sulfate or combinations thereof. Poloxamer 407 is preferred as stabilizer for the present invention. Poloxamer 407 is present in the range from about 0.5% w/w to about 3.0% w/w, preferably in the range from about 1.0% w/w to about 2.0% w/w.

As per one another embodiment of the present invention, a lubricant selected from the group consisting of boric acid, magnesium stearate, sodium stearyl fumarate, micronized poly oxy ethylene glycol, leucine, sodium benzoate, sodium acetate, sodium lauryl sulfate, stearic acid, sodium stearate, sodium oleate, calcium stearate, waxes or combinations thereof. Magnesium stearate is preferred as a lubricant for the present invention and is present in the range from about 0.5% w/w to about 4.5% w/w, preferably in the range from about 1.0% w/w to about 2.5% w/w.

As per one more embodiment of the present invention, a sweetener selected from the group consisting of cyclamate, acesulfame potassium, neo hesperidin dihydrochalcone, monoammonium glycyrrhizinate, saccharin sodium, sucralose, saccharin, aspartame or combinations thereof. Acesulfame potassium is preferred as a sweetener for the present invention. Acesulfame potassium is present in the range from about 0.05% w/w to about 2% w/w, preferably in the range from about 0.30% w/w to about 1.5% w/w.

As per one embodiment of the present invention, a suitable binder for the present invention can be selected from the group consisting of alginic acid, carbomer, ethyl cellulose, gelatin, glucose, guar gum, hydroxy ethyl cellulose, hydroxypropyl cellulose, hydroxypropyl methylcellulose, methylcellulose, polydextrose, polyethylene oxide, and povidone K30 or combinations thereof. Hydroxypropyl cellulose is preferred as a binder for the present invention. Hydroxypropyl cellulose is present in the range from about 0.5% w/w to about 6.0% w/w, preferably in the range from about 1% w/w to about 3% w/w.

As per one another embodiment of the present invention, the orally disintegrating tablet comprising Gabapentin or pharmaceutically acceptable salts thereof is present in the range from about 30% w/w to about 70% w/w, preferably in the range from about 40% w/w to about 60% w/w, a disintegrant present in the range from about 0.5% w/w to about 10% w/w preferably in the range from about 1.5% w/w to about 7.5% w/w and a diluent is present in the range from about 15% w/w to about 55% w/w, preferably in the range from about 30% w/w to about 45% w/w. The orally disintegrating tablet further comprises at least one pharmaceutically acceptable excipient selected from sweeteners, binders, stabilizers, lubricants or combination thereof.

As per preferred embodiment of the present invention, the orally disintegrating tablet comprising Gabapentin or pharmaceutically acceptable salts thereof is present in the range from about 30% w/w to about 70% w/w, preferably in the range from about 40% w/w to about 60% w/w, Crospovidone is present in the range from about 0.5% w/w to about 10% w/w preferably in the range from about 1.5% w/w to about 7.5% w/w and a combination of Microcrystalline cellulose and maize starch is present in the range from about 15% w/w to about 55% w/w, preferably in the range from about 30% w/w to about 45% w/w. The orally disintegrating tablet further comprises at least one pharmaceutically acceptable excipient selected from Acesulfame potassium, Hydroxypropyl cellulose, Poloxamer 407 and Magnesium Stearate.

As per one embodiment of the present invention, the ratio of diluent to disintegrant is in the range of 10:1 to 15:1 and preferably it is in the range of 12:1 to 14:1. As per preferred embodiment of the present invention, the ratio of combination of Microcrystalline cellulose and Maize Starch to Crospovidone is in the range of 10:1 to 15:1 and preferably it is in the range of 12:1 to 14:1. Increase in amount of disintegrant Crospovidone caused a decrease in disintegration time whereas amount of maize starch was changed in different batches and microcrystalline cellulose was kept constant. The ratio of microcrystalline cellulose and maize starch to Crospovidone is optimized in order to achieve fast disintegration of tablet.

As per one embodiment of the present invention, the process for the preparation of orally disintegrating tablets containing Gabapentin or salts thereof, by direct compression method which is one of the most economical methods. The direct compression method is primarily used as it requires fewer processing steps and less equipment. Therefore, the method is potentially less expensive than other methods used in tablet manufacture.

As per one embodiment of the present invention, the disintegrating time of the Gabapentin orally disintegrating tablet is not more than 3 minutes, preferably less than 50 seconds.

As per one embodiment of the present invention, more than 90% of the Gabapentin is released in 15 minutes, preferably more than 90% is released within 10 minutes.

As per one embodiment of the present invention, packaging material for Orally disintegrating tablet of Gabapentin is selected from the Polypropylene Bottle with Silica canister, PP Bottle with Oxygen scavenger, HDPE Bottle, HDPE Bottle with Silica canister, HDPE Bottle with Oxygen scavenger, Alu-Alu Blister. In present invention, the HDPE Bottle with silica canister is preferred as packaging material over other packaging materials as it provided desired physical parameters like appearance of tablets and desired chemical parameters like lesser impurities on storage for 3 months.

As per one embodiment of the present invention, the orally disintegrating tablet of Gabapentin can be effective in the treatment of postherpetic neuralgia and various types of peripheral neuropathic pain, such as painful diabetic neuropathy in adults and partial-onset seizures in paediatrics.

As per one embodiment of the present invention, the orally disintegrating tablet is manufactured by direct compression. Gabapentin, maize starch, microcrystalline cellulose, crospovidone, hydroxypropyl cellulose, Poloxamer 407 and acesulfame potassium were sieved separately through 40 #sieve. Magnesium Stearate was sieve separately through 60 #sieve. Sifted quantity of Gabapentin, maize starch was mixed properly for 15 minutes in blender. To this powder mixture, microcrystalline cellulose, crospovidone, hydroxypropyl cellulose, acesulfame potassium and poloxamer 407 were added and mixed in blender for 15 minutes and then magnesium stearate was added to the pre-lubricated blend and mixed in a blender for 5 minutes. Finally, the lubricated mixture is compressed to form tablets. The prepared tablets were packed in HDPE Bottles with silica canister as it provided good physical parameters and chemical parameters like impurities were found to be lesser as compared to other packaging materials in stability studies for 3 months.

The invention is further illustrated by the following examples, which are by no means intended to limit the scope of the invention but are given by way of illustration.

### Example 1

The Orally disintegrating tablet was made according to the method defined below using the formulation having the ingredients shown in table-I for different dose strengths that are 100 mg, 300 mg, 400 mg, 600 mg and 800 mg of gabapentin:

TABLE I

| Ingredients | % w/w |
|---|---|
| Gabapentin | 54.5 |
| Maize Starch | 15.9 |
| Microcrystalline cellulose | 26.8 |
| Crospovidone | 1.8 |
| Acesulfame potassium | 0.3 |
| Magnesium stearate | 0.7 |
| Total | 100.0 |

### Manufacturing Process:

Gabapentin, microcrystalline cellulose, maize starch, crospovidone and Acesulfame potassium were sieved separately through 40 #sieve. Magnesium Stearate was sieved separately through 60 #sieve. Sifted quantity of gabapentin, maize starch were mixed properly for 15 minutes in blender. To this powder mixture, sifted quantity of crospovidone, microcrystalline cellulose and acesulfame potassium were added and mixed in blender for 15 minutes and then magnesium stearate was added to the pre-lubricated blend and mixed in a blender for 5 minutes. Finally, the lubricated mixture is compressed to form tablets.

Weight variation observed due to poor blend flow. In order to optimize blend flow, it was necessary to increase the concentration of Lubricant.

| Test Parameters | Result |
|---|---|
| Bulk Density (gm/ml) | 0.4475 |
| Tap Density (gm/ml) | 0.6712 |
| Carr Index (%) | 33.33 |
| Hausner Ratio | 1.50 |

### Example 2

The Orally disintegrating tablet was made according to the method defined below using the formulation having the ingredients shown in table II for different dose strengths that are 100 mg, 300 mg, 400 mg, 600 mg and 800 mg of gabapentin:

TABLE II

| Ingredients | % w/w |
|---|---|
| Gabapentin | 54.5 |
| Maize Starch | 15.5 |
| Microcrystalline cellulose | 26.8 |
| Crospovidone | 1.8 |
| Acesulfame potassium | 0 3 |
| Magnesium stearate | 1.1 |
| Total | 100.0 |

### Manufacturing Process:

Gabapentin, microcrystalline cellulose, maize starch, crospovidone, and acesulfame potassium were sieved separately through 40 #sieve. Magnesium Stearate was sieved separately through 60 #sieve. Sifted quantity of Gabapentin, Maize starch were mixed properly for 15 minutes in blender. To this powder mixture, crospovidone, microcrystalline cellulose and acesulfame potassium were added and mixed in blender for 15 minutes and then magnesium stearate to the pre-lubricated blend was added and mixed in a blender for 5 minutes. Finally, the lubricated mixture is compressed to form tablets.

Flow properties increased but tablet lamination observed during compression. In order to remove lamination issue, it was necessary to add Hydroxypropyl Cellulose as a dry binder.

| Test Parameters | Result |
|---|---|
| Bulk Density (gm/ml) | 0.4320 |
| Tap Density (gm/ml) | 0.5865 |
| Carr Index (⁰0) | 26 34 |
| Hausner Ratio | 1.35 |

### Example 3

The Orally disintegrating tablet was made according to the method defined below using the formulation having the ingredients shown in table Ill for different dose strengths that are 100 mg, 300 mg, 400 mg, 600 mg and 800 mg of gabapentin:

TABLE III

| Ingredients | % w/w |
|---|---|
| Gabapentin | 54.5 |
| Maize Starch | 14.58 |
| Microcrystalline cellulose | 26.8 |
| Crospovidone | 1.8 |
| Acesulfame potassium | 0.3 |
| Hydroxypropyl Cellulose | 0.9 |
| Magnesium stearate | 1.1 |
| Total | 100.0 |

### Manufacturing Process:

Gabapentin, microcrystalline cellulose, maize starch, crospovidone, hydroxypropyl cellulose and acesulfame potassium were sieved separately through 40 #sieve. Magnesium Stearate was sieved separately through sieve. Sifted quantity of Gabapentin, maize starch were mixed properly for 15 minutes in blender. To this powder mixture microcrystalline cellulose, crospovidone, hydroxypropyl cellulose and acesulfame potassium were added and mixed in blender for 15 minutes and then magnesium stearate was added to the pre-lubricated blend and mixed in a blender for 5 minutes. Finally, the lubricated mixture is compressed to form tablets.

Still minor tablet lamination was observed. In order to minimize lamination issue, it was necessary to increase concentration of Hydroxypropyl cellulose.

### Example 4

The Orally disintegrating tablet was made according to the method defined below 15 using the formulation having the ingredients shown in table IV for different dose strengths that are 100 mg, 300 mg, 400 mg, 600 mg and 800 mg of gabapentin:

TABLE IV

| Ingredients | % w/w |
|---|---|
| Gabapentin | 54.5 |
| Maize Starch | 13.67 |
| Microcrystalline cellulose | 26.8 |
| Crospovidone | 1 8 |
| Acesulfame potassium | 0.3 |
| Hydroxypropyl Cellulose | 1.8 |
| Magnesium stearate | 1.1 |
| Total | 100.0 |

### Manufacturing Process:

Gabapentin, microcrystalline cellulose, Maize starch, crospovidone, hydroxypropyl cellulose and acesulfame potassium were sieved separately through sieve. Magnesium Stearate was sieved separately through 604 sieve. Sifted quantity of gabapentin, maize starch were mixed properly for 15 minutes in blender. To this powder mixture, microcrystalline cellulose, crospovidone, hydroxypropyl cellulose and acesulfame potassium were added and mixed in blender for 15 minutes and then magnesium stearate was added to the pre-lubricated blend and mixed in a blender for 5 minutes. Finally, the lubricated mixture is compressed to form tablets.

Disintegration time observed higher side than usual. In order to decrease disintegration time, it was necessary to increase concentration of disintegrant.

### Example 5

The Orally disintegrating tablet was made according to the method defined below using the formulation having the ingredients shown in table V for different dose strengths that are 100 mg, 300 mg, 400 mg, 600 mg and 800 mg of gabapentin:

TABLE V

| Ingredients | % w/w |
|---|---|
| Gabapentin | 54.5 |
| Maize Starch | 12.76 |
| Microcrystalline cellulose | 26.8 |
| Crospovidone | 2.7 |
| Acesulfame potassium | 0.3 |
| Hydroxypropyl Cellulose | 1.8 |
| Magnesium sterate | 1.1 |
| Total | 100.0 |

### Manufacturing Process:

Gabapentin, microcrystalline cellulose, maize starch, crospovidone, hydroxypropyl cellulose and acesulfame potassium were sieved separately through sieve. Magnesium Stearate was sieve separately through 60 #sieve. Sifted quantity of gabapentin, maize starch were mixed properly for 15 minutes in blender. To this powder mixture Microcrystalline cellulose, crospovidone, microcrystalline cellulose, hydroxypropyl cellulose and acesulfame potassium were added and mixed in blender for 15 minutes and then magnesium stearate was added to the pre-lubricated blend and mixed in a blender for 5 minutes. Finally, the lubricated mixture is compressed to form tablets.

All the physical parameters of tablets were found to be satisfactory but chemical parameters of tablets were found not satisfactory after 1-month stability data of Related Substances. In order to stabilize formulation during stability, it was necessary to add stabilizer as a Poloxamer 407.

| Test Parameters | Result |
|---|---|
| Bulk Density (gm/ml) | 0 4130 |
| Tap Density (gm/ml) | 0.5531 |
| Carr Index (%) | 25.32 |
| Hausner Ratio | 1.33 |

### Example 6

The Orally disintegrating tablet was made according to the method defined below using the formulation having the ingredients shown in table VI for different dose strengths that are 100 mg, 300 mg, 400 mg, 600 mg and 800 mg of gabapentin:

TABLE VI

| Ingredients | % w/w |
|---|---|
| Gabapentin | 54.5 |
| Maize Starch | 10.95 |
| Microcrystalline cellulose | 26.73 |
| Crospovidone | 2.73 |
| Acesulfame potassium | 0.33 |
| Poloxamer 407 | 1.81 |
| Hydroxypropyl Cellulose | 1.81 |
| Magnesium stearate | 1.09 |
| Total | 100.0 |

### Manufacturing Process:

Gabapentin, maize starch, microcrystalline cellulose, crospovidone, hydroxypropyl cellulose, poloxamer 407 and acesulfame potassium were sieved separately through 40 #sieve. Magnesium Stearate was sieve separately through 60 #sieve. Sifted quantity of gabapentin, maize starch were mixed properly for 15 minutes in blender. To this powder mixture, microcrystalline cellulose, crospovidone, hydroxypropyl Cellulose, acesulfame potassium and poloxamer 407 were added and mixed in blender for 15 minutes and then magnesium stearate was added to the pre-lubricated blend and mixed in a blender for 5 minutes. Finally, the lubricated mixture is compressed to form tablets The prepared tablets were packed in HDPE Bottles with silica canister as it provided good physical parameters and chemical parameters like impurities were found to be lesser as compared to other packaging materials in stability studies for 3 months.

All the physical and chemical parameters of tablets were found satisfactory after 3-month stability data as per below results:

| Test Parameters | Result |
|---|---|
| Bulk Density (gm/ml) | 0.4075 |
| Tap Density (gm/ml) | 0.5314 |
| Carr Index (%) | 23.31 |
| Hausner Ratio | 1.30 |

### Example 7: The Dissolution Profile of the Tablet Prepared According to Example 6

The conditions of dissolution are as following:
Product Name: Gabapentin 300 mg orally disintegrating tablet
Media: 0.1 N HCl
Apparatus & RPM: Apparatus II (Paddle)
Rate of rotation: 50 RPM
Volume: 900 ml
Temperature: 37 °C
Detection: High performance liquid chromatography equipped with UV/PDA Detector at 210 nm

The orally disintegrating tablet of Gabapentin was tested for its dissolution profile measured in 900 mL of 0.1 N HCA at 50 RPM in USP 11 (Paddle) apparatus and active ingredient of the tablet is released in more than 90% in 10 minutes.

Example 8: The orally disintegrating tablets prepared according to example 6 were subjected to a stability study of 25 °C/60% RH for 3 months. The stability study was performed for 300 mg dose strength of Gabapentin. Results are tabulated below.

### Gabapentin 300 mg Orally Disintegrating Tablet

| | | | | Exp -6 | |
|---|---|---|---|---|---|
| | | | | 3 Months | |
| | | Exp-6 | | 25 °C/60% | |
| Parameters | Specification | Initial | | RH | |
| Hardness | 3 ON-100N | 46N | | 38N | |
| | (Target 45N) | | | | |
| Average weight | 550.0 mg ± 5% | 552.4 | mg | 551.4 | mg |
| | 522.5 mg-577.5 mg | | | | |
| Thickness | 4.50 + 0.30 mm | 4.53 | mm | 4.52 | mm |
| | (4.20 mm to 4.80 mm) | | | | |
| Friability | Not more than 1.0% | 0.29% | | 0.29% | |
| Disintegration | Not more than 3 | 14 | sec | 22 | sec |
| time | minutes | | | | |

| | | | |
|---|---|---|---|
| Dissolution | NLT 75% (Q) labelled | 98.9 | 97.6 |
| | amount of Gabapentin | | |
| | should be dissolve in | | |
| | 15 minutes. | | |
| Assay (%) | 95.0-105.0% | 100.0 | 101.4 |

Impurities (%)
RS-A

| | | | |
|---|---|---|---|
| Impurity A | Not more than 0.4% | 0.051 | 0.0572 |
| Single maximum | Not more than 0.1% | ND | ND |
| unknown impurity | | | |

RS-B

| | | | |
|---|---|---|---|
| Single maximum | Not more than 0.05% | ND | ND |
| unknown impurity | | | |
| Total impurities for | Not more than 1.0% | 0.051 | 0.0572 |
| RS-A and RS-B | | | |

1. An orally disintegrating tablet of Gabapentin for oral administration comprising: a) Gabapentin or pharmaceutically acceptable salts thereof, present in the range of about 30% w/w to about 70% w/w, preferably in the range from about 40% w/w to about 60% w/w;
b) at least one diluent;
c) at least one disintegrant present in the range from about 0.5% w/w to about 10.0% w/w, preferably in the range from about 1.5% w/w to about 7.5% w/w; and
d) at least one stabilizer present in the range from about 0.5% w/w to about 3.0% w/w, preferably in the range of about 1.0% w/w to about 2.0% w/w.

2. The orally disintegrating tablet according to claim 1, wherein the diluent is selected from the group consisting of spray-dried lactose, dicalcium phosphate, dextrates, microcrystalline cellulose, dextrose, maize starch, fructose, sorbitol, pregelatinized starch, starch, xylitol, sucrose, sorbitol, maltodextrin, maltose, mannitol or combinations thereof.

3. The orally disintegrating tablet according to claim 2, wherein the diluent is microcrystalline cellulose present in the range from about 15% w/w to about 50% w/w, preferably in the range from about 20% w/w to about 40% w/w.

4. The orally disintegrating tablet according to claim 2, wherein at least 30% of the total weight of microcrystalline cellulose has an average particle size in the range from about 70 μm to about 100 μm, preferably in the range from about 90 μm to about 100 μm and the bulk density is in the range from about 0.26 g/ml to about 0.34 g/ml, preferably in the range from 0.30 g/ml to about 0.33 g/ml.

5. The orally disintegrating tablet according to claim 2, wherein at least 70% of the total weight of microcrystalline cellulose has a particle size lesser than 75 μm, preferably lesser than 70 μm and the bulk density is in the range from about 0.10 g/cm³ to about 0.15 g/cm³, preferably from about 0.11 g/cm³ to about 0.14 g/cm³.

6. The orally disintegrating tablet according to claim 2, wherein the diluent is Maize Starch present in the range from about 5% w/w to about 30% w/w, preferably in the range from about 7% w/w to about 25% w/w.

7. The orally disintegrating tablet according to claim 1, wherein the diluent is the combination of microcrystalline cellulose and Maize starch present in the range from about 15% w/w to about 55% w/w, preferably in the range from about 30% w/w to about 45% w/w.

8. The orally disintegrating tablet according to claim 1, wherein the disintegrant is selected from the group consisting of modified starches, cross-linked polyvinylpyrrolidone, croscarmellose sodium, soy polysaccharide, cross-linked alginic acid, gellan gum, calcium silicate or combinations thereof.

9. The orally disintegrating tablet according to claim 8, wherein the disintegrant is Crospovidone.

10. The orally disintegrating tablet according to claim 1, wherein the stabilizer is selected from the group consisting of Poloxamers, Amino acid-based stabilizers, sodium alginate, sodium carboxymethyl cellulose (CMC), guar gum, locust bean gum, carrageenan, gelatin, and pectin, Povidone, Polyvinyl alcohol, Sodium lauryl sulfate or combinations thereof.

11. The orally disintegrating tablet according to claim 10, wherein the stabilizer is Poloxamer 407.

12. The orally disintegrating tablet according to claim 1, wherein pharmaceutical composition further comprises at least one or more pharmaceutically acceptable excipients are selected from the group consisting of binders, sweeteners, and lubricants or combinations thereof.

13. The orally disintegrating tablet according to claim 12, wherein the lubricant is selected from the group consisting of boric acid, magnesium stearate, Sodium Stearyl fumarate, micronized poly oxy ethylene glycol, leucine, sodium benzoate, sodium acetate, sodium lauryl sulfate, stearic acid, sodium stearate, sodium oleate, Poloxamer 407, calcium stearate, waxes or combinations thereof.

14. The orally disintegrating tablet according to claim 12, wherein the lubricant is Magnesium Stearate is present in the range from about 0.5% w/w to about 4.5% w/w, preferably in the range from about 1.0% w/w to about 2.5% w/w.

15. The orally disintegrating tablet according to claim 12, wherein the binder is selected from the group consisting of alginic acid, carbomer, ethyl cellulose, gelatin, glucose, guar gum, hydroxy ethyl cellulose, hydroxypropyl cellulose, hydroxypropyl methylcellulose, methylcellulose, polydextrose, polyethylene oxide, and Povidone K30 or combinations thereof.

16. The orally disintegrating tablet according to claim 12, wherein the binder is hydroxypropyl cellulose present in the range from about 0.5% w/w to about 6.0% w/w, preferably in the range from about 1% w/w to about 3% w/w.

17. The orally disintegrating tablet according to claim 12, wherein the sweetener is selected from the group consisting of cyclamate, acesulfame potassium, neo hesperidin dihydrochalcone, monoammonium glycyrrhizinate, saccharin sodium, sucralose, saccharin, aspartame or combinations thereof.

18. The orally disintegrating tablet according to claim 12, wherein the sweetener is acesulfame potassium present in the range from about 0.05% w/w to about 2% w/w, preferably in the range from about 0.30% w/w to about 1.5% w/w.

19. (canceled)

20. The orally disintegrating tablet according to claim 1, wherein the ratio of a diluent to disintegrant is in the range of 10:1 to 15:1 and preferably in the range of 12:1 to 14:1.

21. The orally disintegrating tablet according to claim 1, wherein the orally disintegrating tablet is manufactured by the direct compression method comprising the steps of:
(a) Sieving separately Gabapentin, microcrystalline cellulose, maize starch, crospovidone, Hydroxy Propyl Cellulose, Poloxamer 407, and Acesulfame potassium through 40 #sieve and magnesium Stearate separately through 60 #sieve;
(b) Mixing of the previously sifted quantity of gabapentin and Maize starch for 15 minutes in a blender;
(c) Blending the sifted quantity of microcrystalline cellulose, crospovidone, hydroxy propyl cellulose, poloxamer 407, and Acesulfame potassium in the blender with step-(b) for 15 minutes in blender;
(d) Adding magnesium stearate to the pre-lubricated blend of step-(c) in a blender for 5 minutes;
e) Compressing the resulting mixture into a tablet; and
(f) packing of the tablet into HDPE Bottles with silica canister.

22-23. (canceled)

24. The orally disintegrating tablet according to claim 1, wherein the packaging material of the container is selected from the Polypropylene Bottle with Silica canister, PP Bottle with Oxygen scavenger, HDPE Bottle, HDPE Bottle with Silica canister, HDPE Bottle with Oxygen scavenger, Alu-Alu Blister.

25. (canceled)
